(19) United States
(12) Patent Application Publication
(10) Pub. No.: US 2010/0228180 A1
(43) Pub. Date: Sep. 9, 2010

(54) Power Source Electrode Treatment Device

(75) Inventors: Dalia Jayes, Modiin (IL); Mordechay Moshkovich, Ra'anana (IL); Zvi Nitzan, Zofit (IL)

Correspondence Address: KENYON & KENYON LLP, 1500 K STREET N.W., SUITE 700, WASHINGTON, DC, 20005, US

(73) Assignee: POWER PAPER LTD, Petah Tikva (IL)

(21) Appl. No.: 12/223,178

(22) PCT Filed: Jan. 25, 2007
(86) PCT No.: PCT/IL2007/000093

§ 371 Date: Aug. 25, 2008

Related U.S. Application Data
(60) Provisional application No. 60/762,137, filed on Jan. 26, 2006.

(51) Int. Cl. A61N 1/30

(52) U.S. Cl. 604/20

(57) ABSTRACT

The present invention provides a powered treatment device comprising at least one power source for supplying electrical energy to the device, and wherein at least one component of the power source is also configured as a power source treatment device electrode to facilitate electrical contact with a body region. In an embodiment wherein one component of the power source is configured as a power source treatment device electrode, the powered treatment device may further comprise at least one non-power source device electrode of opposite polarity from the power source treatment device electrode, wherein the at least one non-power source device electrode is electrically coupled to the at least one power source to facilitate electrical contact with a body area.

### FIELD OF THE INVENTION

The present invention relates to a treatment device for treatment of any suitable body region. Moreover, the present invention is of a device including a power source, wherein at least one of the power source components is also configured as a treatment device electrode.

### BACKGROUND OF THE INVENTION

The techniques of electrotransportation such as iontophoresis, electroporation and electroosmosis are well documented in the art and are used for administration of drugs and cosmetics. These techniques have been incorporated into many devices, including transdermal delivery devices, such as the dermal patch. Typically, transdermal delivery devices, such as active patches described in the background art feature a power source, a medical electrode and a counter electrode, wherein the medical and counter electrodes are electrically connected to the power source for aiding transdermal delivery. Dermal patches featuring a galvanic couple, which powers the drug delivery or a plurality of galvanic couples connected to each other are also disclosed in the art, such as in U.S. Pat. No. 6,421,561 to Morris and US Patent Application Publication No. 20050004508 to Sun et al.

Latent deficiencies of the transdermal delivery devices of the background art include insufficient current and voltage resulting from a galvanic couple, which can limit penetration depths of active substances and high production costs of multi component devices. In addition, resistance between the power source and active electrode can result in lower current density at the active electrode.

There is thus a need for, and it would be highly advantageous to have, a treatment device, such as a dermal/transdermal delivery device, with a power source configured to supply sufficient current to the device, but wherein the device includes minimal components. Moreover, it would be desirable to have a treatment device, wherein at least one of the power source components is also configured as one of the treatment device electrodes. Further, it would be desirable to have such a treatment device, which facilitates greater current density at the active electrode. Still further, it would be desirable to have a treatment device, which includes minimal components for optimal function, in order to facilitate more simple production and a cheaper product.

### BRIEF DESCRIPTION OF THE DRAWINGS

With reference now to the drawings in detail, it is stressed that the particulars shown, are by way of example and for the purposes of illustrative discussion of embodiments of the present invention, and are presented for providing what is believed to be the most useful and readily understood description of the principles and conceptual aspects of the invention. In this regard, no attempt is made to show structural details in more detail than is necessary for a fundamental understanding of the invention, the description taken with the drawings making apparent to those skilled in the art how the several forms of the invention may be embodied in practice.

In the drawings:

FIG. 1a shows a schematic view of a powered treatment device according to one embodiment of the present invention;

FIG. 1b shows a schematic view of a powered treatment device with an extended power source component according to one embodiment of the present invention;

FIG. 1c shows a schematic view of a powered treatment device wherein the power source poles are in a coplanar configuration according to one embodiment of the present invention;

FIG. 2a shows a schematic view of a powered treatment device according to one embodiment of the present invention;

FIG. 2b shows an embodiment of a powered treatment device wherein an extension of a power source current collector is configured as a power source device electrode according to one embodiment of the present invention;

FIG. 2c shows an embodiment of a powered treatment device wherein an extension of a power source current collector is configured as a power source device electrode and wherein the power source poles are in a coplanar configuration;

FIG. 2d shows an embodiment of a powered treatment device wherein an extension of a power source current collector is configured as a power source device electrode;

FIG. 3 shows a schematic view of a power source according to one embodiment of the preset invention;

FIG. 4 shows a schematic view of one non-limiting example of a power source which can be used in the device of the present invention according to an embodiment of the present invention;

FIG. 5 shows a schematic view of a powered treatment device including a conductive interfacing media according to one embodiment of the present invention;

FIG. 6a shows a schematic view of a powered treatment device wherein two power source components are configured as treatment device electrodes;

FIG. 6b shows a schematic view of a powered treatment device wherein the power source poles are in a coplanar configuration and two power source components are configured as treatment device electrodes; and

FIG. 7 shows a flow chart of a method of using the powered treatment device of the present invention according to one embodiment of the present invention.

### DETAILED DESCRIPTION OF THE PREFERRED EMBODIMENTS

The present invention is of a powered treatment device. Moreover, the present invention is of a powered treatment device configured for treatment of a body region. Still further the present invention is of powered treatment device configured for at least one of electrical stimulation and promotion of active compound delivery. Further, the present invention is of a treatment device featuring a power source for powering the device and wherein at least one of the power source components is also configured as a device electrode. The power source may comprise components such as at least one positive pole, an electrolyte, at least one negative pole, at least one current collector and at least one terminal. In some embodiments the power source poles are in a cofacial orientation. In some embodiments the power source poles are in a coplanar orientation.

In one embodiment two of the power source components are configured as both treatment device electrodes.

In an alternative embodiment one of the power source components is configured as a treatment device electrode and the treatment device further comprises a non-power source treatment device electrode. In such an embodiment the power source component treatment device electrode may be in electrical contact with the non-power source device electrode. In some embodiments, the treatment device components are disposed on a substrate base layer.

In some embodiments the powered treatment device features a conductive interface media coupled to at least one of the device electrodes. The term ‘conductive interface media’ includes any suitable conducting means, electrically conductive and/or ionically conductive, which facilitates a conductive interface between the powered treatment device and body area. One non-limiting example is a hydrogel. Optionally, conductive interface media can be disposed in a holding means. Optionally, device can include an active substance.

In an additional embodiment, the present invention provides a method of production of the device.

In a still further embodiment, the present invention provides uses of the powered treatment device. Uses include, but are not limited to acne treatment, sebum regulation, rosacea, age spots, treatment of pores, dermatitis, skin and nail viral, fungal and bacterial infections, onychomycosis, disorders of the hair follicles and sebaceous glands, scaling disease, dark rings under the eyes, scars, wounds, cellulite treatment, skin and tooth whitening, pigmentation disorders, sun damaged skin, fine facial lines, laugh lines, aging skin, dry skin, wrinkles, puffy eyes, lifting skin, folliculitis, dermatitis, psoriasis, warts, benign tumors, malignant tumors, pain management, bone healing, facilitating muscle contraction, promoting metabolic processes, increasing blood flow, hair growth disorders, treating hyperhidrosis, body decoration, vaginal candidiasis and vaginosis, genital herpes, as a drug delivery system for delivering any suitable drug or active ingredient to any suitable body region and combinations thereof.

The present invention overcomes deficiencies of devices of the background art, wherein the powered treatment device of the present invention is configured with minimal device elements, which may facilitate better electrical contact between power source and device electrode and optimal current density at the active electrode for target penetration depth. Further, the present invention may facilitate a fully integrated treatment device and a more facile and cheaper production process.

Before explaining at least one embodiment of the invention in detail, it is to be understood that the invention is not limited in this application to the details of construction and the arrangement of the components set forth in the following description or illustrated in the drawings. The invention is applicable to other embodiments of being practiced or carried out in various ways. Also, it is to be understood that the phraseology and terminology employed herein is for the purpose of description and should not be regarded as limiting.

The principles and operation of powered treatment devices according to the present invention may be better understood with reference to the figures. The figures show exemplary embodiments of the present invention and are not limiting.

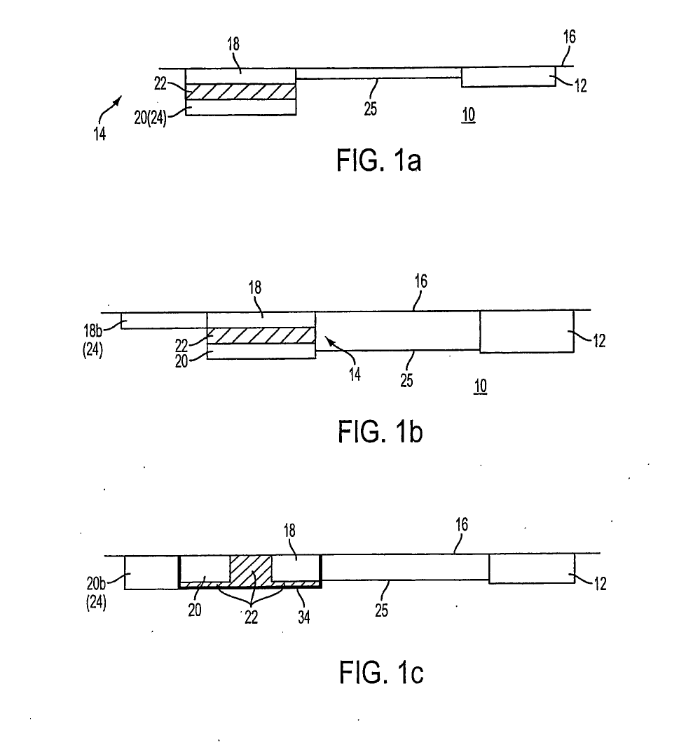

FIG. 1a shows a schematic view of one embodiment of a powered treatment device 10. As can be seen from FIG. 1a, device 10 includes a non-power source treatment device electrode 12 and a power source 14 supported on a base layer substrate 16 in spaced relation to each other. Power source can be any suitable power source, which includes a positive pole 18 and a negative pole 20 as defined by the electropotential values of the pole elements, with an electrolyte 22 disposed between the positive and negative poles. FIG. 1a shows one configuration of poles 18, 20 and electrolyte 22, however this configuration is not intended to be limiting and any suitable configuration may be used, such as wherein negative pole 20 is disposed above electrolyte 22 and positive pole 18 is disposed below.

In some embodiments, treatment device 10, including device components, is thin and flexible, which may suit the contour of a body area of a subject. Treatment device 10 may be of any size, color and shape suitable for application to a desired body area. In some embodiments, treatment device is a patch. The thickness of patch 10 may be up to 10 mm to ensure flexibility, but may be thicker, depending on the application. The thickness of the patch may also be dependent upon the type of material used and the flexibility of that material. In some embodiments patch 10 may be disposable. In some embodiments patch 10 may be reusable. Treatment device 10 may be stable to a wide range of temperatures and humidity.

Treatment with the device of the present invention may be beneficial in all body areas. In an embodiment, wherein device is thin, flexible and versatile in shape and form, the device of the present invention can be designed to fit any area of the body and to have any desirable size, according to the area to be treated.

The device of the present invention may be used to deliver almost any active substance. It is understood that the invention may be used for delivery of a wide range of dosages of active substance over a desired duration of time.

Base layer substrate 16 may optionally be manufactured from any suitable material, which can accommodate the powered treatment device components. Suitable materials include, but are not limited to woven material, non-woven material, polymers, conducting material, non-conducting material, paper, cardboard, plastic, synthetic materials, natural materials, fabric, metals, wood, glass, Perspex, or a combination thereof. In some embodiments, the material of base member 16 is a non-conductive material. In some embodiments, base layer substrate 16 is made from vinyl material or polyester. Optionally, base layer substrate 16 can be made up of a plurality of materials, which can be stacked or connected in a co-planar way by any suitable attachment means. In some embodiments, base layer substrate 16 is made up of one continuous piece of material.

Optionally, substrate base layer 16 may readily facilitate attachment of the patch 10 to a desired body area. Attachment mechanisms may include but are not limited to conductive adhesive, adhesive strip, suction cups and/or any combinations thereof. The patch may be attached to the body area by, for example, the frame of the substrate and/or other attachment mechanisms.

Electrode 12 can be made of any suitable material, such as zinc, copper, manganese dioxide, iron, magnesium, silicon, sodium, silver, silver/silver chloride, carbon, graphite, platinum, nickel, gold, lithium or a combination thereof. Electrode 12 can be referred to herein as ‘non-power source device electrode’. Electrode 12 can optionally be an active electrode/medical electrode or a counter electrode. As used herein the term ‘active electrode’ refers to the electrode, under, on or in which the active substance to be delivered is disposed. In the embodiment shown in FIG. 1a, electrode 12 is the counter electrode. The definition of which electrode is the active electrode is dependent on the electrode material and on the charge of the active component to be delivered. In an embodiment wherein no active component is to be delivered, defining the main electrode is dependent on the direction of the flow of electricity. In some embodiments, wherein electrode 12 is the active electrode, electrode can be made from an insoluble electroactive substance, which has therapeutic or biological properties, such as zinc, silver, magnesium, lithium and copper. Optionally, electrode 12 can be made by any suitable technique. In some embodiments, electrode is made by a suitable printing technique. In some embodiments electrode 12 can be printed onto base layer substrate or alternatively electrode 12 can be printed onto an additional base layer (not shown in FIG. 1a) which can be attached to base layer substrate 16. Electrode 12 can be disposed in any suitable way on substrate 16 in spaced relation to power source 14 and electrically connected 25 to power source 14 in any suitable way. In some embodiments, electrode 12 can be integrally formed with or disposed on power source 14. In some embodiments electrode 12 may be printed in any suitable way on power source 14.

Optionally, in an embodiment wherein alternating current is used or when two compounds with opposite charges are to be delivered, both anode and cathode electrodes can be active electrodes. Power source 14 may be any suitable power source. According to one embodiment of the present invention, power source 14 may be an electrochemical cell. In some embodiments, power source 14 may be thin and flexible. In one embodiment, power source 14 may be disposable. In an alternative embodiment, power source 14 may be rechargeable. The term “power source” as used herein includes, but is not limited to, any suitable arrangement of components in which chemical energy is converted to electric energy by a spontaneous electron transfer reaction. The term includes cells with non-spontaneous reactions, galvanic cells, galvanic couples, electrolytic cells, and/or a combination thereof.

In some embodiments power source 14, can provide a direct current electrical potential. In some embodiments, the current and or voltage supplied by the power source is fixed and cannot be adjusted by a user. In yet another embodiment, the electrical potential may be adjusted.

Optionally, power source 14 may be a single electrochemical cell. However, power source 14 need not be limited to one cell, but may include a plurality of connected cells, electrochemical cells, galvanic cells, a plurality of batteries, and/or electronics configured to increase, control, and change phase of the supplied electric current. In some embodiments, electrochemical cell 14 in device provides electrical potential (voltage) to the desired body area of the subject.

The power source 14 may optionally be located in any suitable position on the device.

Power source 16 may include a negative pole 20 and a positive pole 18, and an electrolyte 22 disposed between the negative pole 20 and positive pole 18. Positive pole 18 may be made from any suitable material, such as but not limited to lead dioxide, lead oxide, nickel hydroxide, lithium manganese oxide, manganese dioxide, silver oxide and nickel oxide. Negative pole may be made from any suitable material, such as but not limited to zinc, lead, cadmium, iron, silver, gold, magnesium, copper, aluminum, carbon and graphite. Electrolyte 22 may be any suitable electrolyte. In some embodiments, one of negative pole 20 or positive pole 18 is also configured as active/main electrode or counter electrode of treatment device 10, to be referred to herein as ‘power source treatment device electrode’ 24. In FIG. 1a, power source device electrode 24 is shown as power source negative pole 20, however this is for illustration purposes only. In some embodiments power source 14 can be disposed on base layer substrate, such that positive pole electrode 18 may be power source device electrode 24.

In one embodiment shown in FIG. 1b, the power source can be made, such as by a printing technique, such that the region of power source pole, which is to be configured as power source device electrode is an extended region of the pole. Part or all of the extension 18b can be used as power source device electrode. In FIG. 1b power source positive electrode 18 includes an extended region 18b, which is configured as power source device electrode 24. In the embodiments shown in FIG. 1a and FIG. 1b, power source 14, includes positive pole 18 and negative pole 20 in a cofacial arrangement. Optionally, power source 14 may include poles 18 and 20 in a coplanar configuration as shown in FIG. 1c. FIG. 1c shows battery poles 18 and 20 in a coplanar arrangement with an electrolyte 22 between the poles. An extension 20b of the pole 20 may be configured as a power source device electrode 24. The power source may be covered by a suitable cover 34 and the non-power source electrode 12 may be electrically connected to the power source.

In one embodiment, the electroactive insoluble material of the power source pole, which is also configured as a power source treatment device electrode, is formulated in order to be biocompatible. Depending on the type of power source being used, the pole electrodes can comprise inks or foils or any other suitable form of insoluble electroactive pole material.

Due to the power source device electrode 24 being an integral part of the power source 14, power source device electrode 24 is configured to facilitate optimal electrical contact between the power source device electrode 24 and the power source 14, eliminating the need for additional connection means. Therefore, the device of the present invention 10 has improved connection compared to devices described in the art wherein both electrodes are connected by connection means to a power source.

Power source 14 can be of any suitable size and shape. In some embodiments, such as described in FIGS. 1a, 1b and 1c, the treatment device of the present invention 10 features only one non-power source device electrode and is thus configured that it may facilitate a larger power source 14 compared to a similar sized treatment device of the art which includes two non-power source device electrodes. A larger power source may facilitate a higher current density. In one embodiment, the treatment device may be smaller than devices with two non-power source electrodes.

In some embodiments, non-power source device electrode 12 is connected to battery 14 by any suitable connection means 25, such as electrical conduction means/media. Examples of connection means include, but are not limited to wiring, conductive ink, conductive via, printed connection means, soldered connection means, connection means attached by UV, adhesive connection means, conductive adhesive, conductive adhesive tape and a combination thereof.

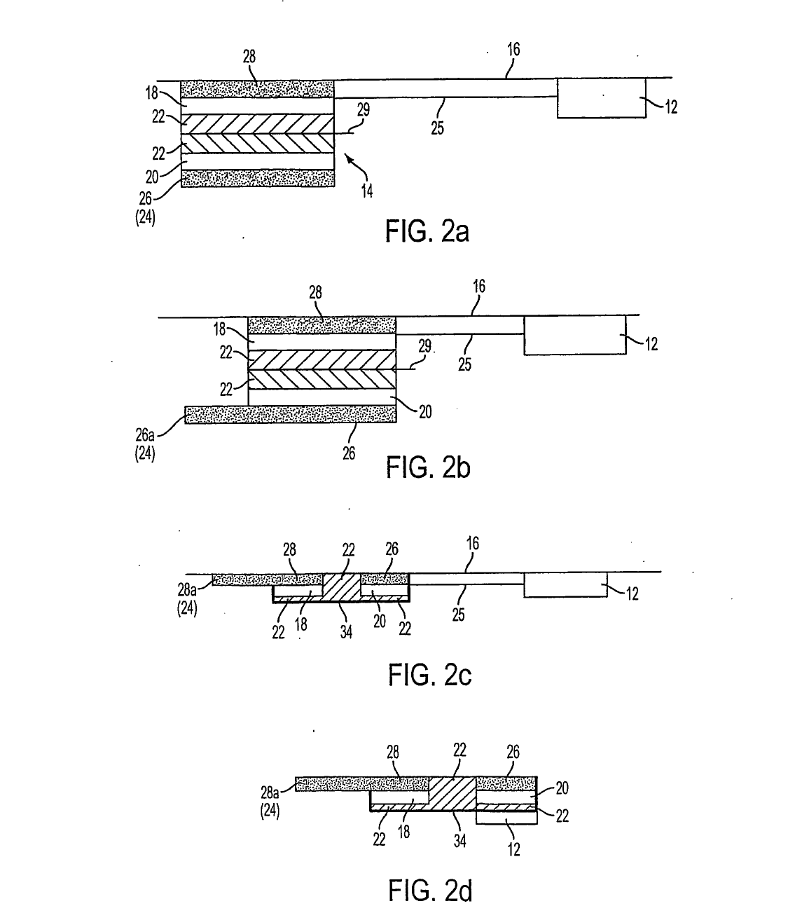

In an alternative embodiment shown in FIG. 2a, power source may include at least one current collector. In one embodiment, power source may include a positive current collector 28, on which is disposed a positive pole 18, a negative pole 20 and a negative current collector 26 and at least one electrolyte layer 22 disposed on the positive pole and on the negative pole. In some embodiments power source may include a separator 29. Separator 29 may be any suitable separator made by any suitable technique including, but not limited to a printable separator. In some embodiments, power source 14 may only include one current collector. Current collectors 26, 28 may be made from any suitable conductive material, such as, but not limited to carbon, graphite, silver, platinum, nickel, copper or gold or combinations thereof. In some embodiments current collectors 26, 28 are graphite or carbon based layers, which can be printed or applied in any suitable way to cell 14. Examples of graphite and carbon based layers include graphite or carbon webs, sheets, inks and cloth.

In one embodiment, power source current collector 26 (as shown in FIG. 2a) or 28 depending how power source is disposed on base layer 16, can be configured as power source device electrode 24. The power source 14 shown in FIG. 2a illustrates a cofacial configuration of pole electrodes 18 and 20. The embodiment of the invention described for FIG. 2a is not intended to be limited to a cofacial orientation, but is also applicable to a coplanar configuration of poles 18 and 20.

FIG. 2b shows an embodiment of a device wherein an extension of power source current collector 26a or 28a is configured as power source device electrode 24.

FIG. 2c shows an embodiment of a device wherein an extension of power source current collector 26a or 28a is configured as power source device electrode 24 and the power source poles 18, 20 are in a coplanar configuration.

FIG. 2d shows an embodiment of a device wherein an extension 28a of power source current collector 28 is configured as a power source device electrode, the power source poles 18, 20 are in a coplanar configuration and a non-power source electrode 12 may be disposed on the power source in electrical contact with pole 20. Any suitable electrical contact means may be used.

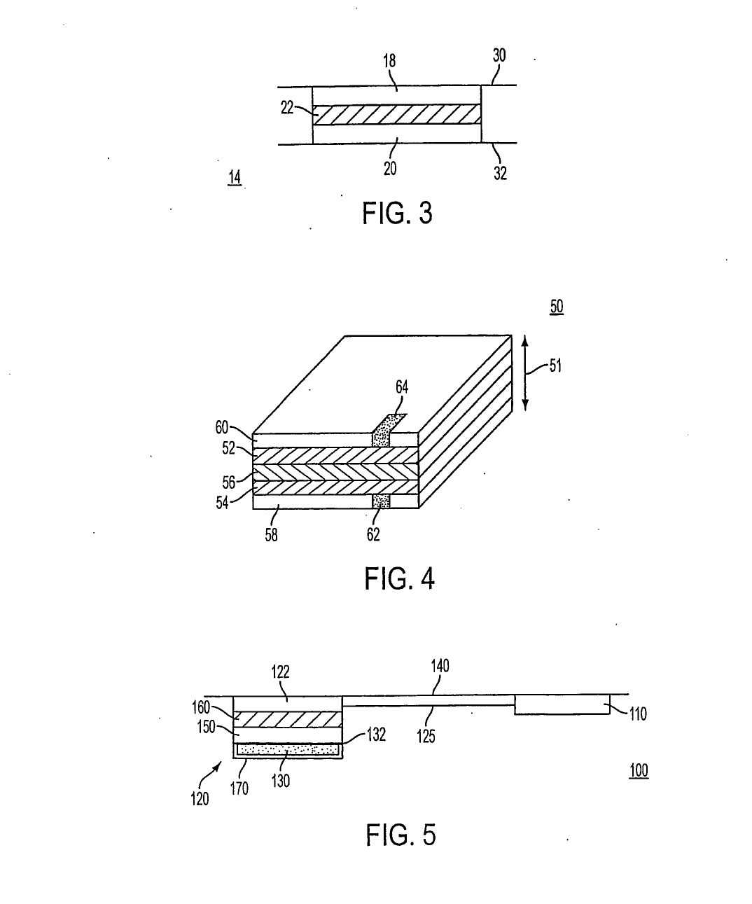

In some embodiments, power source 14 includes at least one substrate base layer on which the power source components are disposed. FIG. 3 shows one embodiment of a power source 14, which may include a positive pole 18, an electrolyte 22 and a negative pole 20. In this embodiment the positive pole 18 is disposed on the power source base layer 30 and the negative pole 20 is disposed on the power source base layer 32. In some embodiments power source base layer substrate 30 and 32 may be constructed from different materials or the same materials.

In some embodiments the power source device electrode 24 is configured to be directly exposed for contact with a body area. In some embodiments, the power source device electrode 24 is partially or completely covered by a power source base layer substrate 32. In some embodiments the power source base layer substrate 32 may be the same as the power source cover 34 shown in FIG. 1c. The power source base layer substrate 30 which is not configured to be in contact with a body area may be comprised of a non-conductive material, which may be semi-porous, such as but not limited to paper and polyester. In some embodiments the power source base layer substrate 30 is the same as the treatment device base layer substrate 16 (as shown in FIGS. 1 and 2) and the power source 14 can be applied directly to the treatment device base layer substrate. In some embodiments such as, but not limited to wherein a power source has been previously made and is for disposing on a treatment base layer substrate, the power source base layer substrate 32, on which is disposed the power source device electrode 24 is configured to contact a body area. In such an embodiment, the power source base layer substrate 32 may be made from a conductive material. The conductive material may facilitate electrical contact between the power source device electrode 24 and the body area. In an alternative embodiment, the power source base layer substrate 32 may be made from a non-conductive material, which includes at least one conductive via. The at least one via may include a conductive means, such as a conductive adhesive. In some embodiments, the power source base layer substrate 32 is comprised of a biocompatible material. One non-limiting example of a non-conductive biocompatible material which can be used is silk.

FIG. 4 illustrates a schematic representation of an exemplary power source 50 of the prior art, which can be used in accordance with an embodiment of the invention. Power source 50 may be thin and flexible. In the embodiment of FIG. 4, the power source is depicted as an electrochemical cell. The thickness 51 of the electrochemical cell 50 may be up to about 4 mm, more preferably up to about 2 mm and most preferably up to about 1 mm.

Electrochemical cell 50 includes a positive pole layer 52, a negative pole layer 54, and an electrolyte layer 56 interposed therebetween. In some embodiments, electrochemical cell 50 includes one or more additional conductive layers 58 and 60 to improve the conductivity of pole layers 52 and 54. Suitable conductive layers 58 and 60 are preferably made from any suitable conductive material, such as carbon, graphite, silver, platinum or gold or combinations thereof. Preferably conductive layers (current collectors) 58 and 60 are graphite or carbon based layers, which can be printed or applied in any suitable way to cell 50. Examples of graphite and carbon based layers include graphite or carbon webs, sheets, inks and cloth. Preferably, electrochemical cell includes negative terminals 62 and positive terminals 64, which are in contact with the corresponding pole layer 54 and 52 or with the corresponding conductive layer 58 and 60 or both. Terminals are made of any suitable material such as, but not limited to, graphite or metal and are preferably applied to cell 50 by a suitable printing technology. Terminals may be located in any desired location of cell 50 and may acquire any suitable shape and size, depending on the specific application. Optionally, terminals may protrude from the surface of cell 50.

By way of example, a suitable electrochemical cell 50 is described in U.S. Pat. Nos. 5,652,043, 5,897,522, and 5,811,204, each of which are incorporated herein by reference in their entireties. Briefly, the electrochemical cell described in the above-identified U.S. patents is an open liquid state, electrochemical cell, which can be used as a primary or rechargeable power source for various miniaturized and portable electrically powered devices of compact design. In one embodiment, a preferable electrochemical cell 50 may comprise a first layer of insoluble negative pole 54, a second layer of insoluble positive pole 52, and a third layer of aqueous electrolyte 56 disposed between the first 54 and second 56 layers and may include (a) a deliquescent material (not shown) for keeping the open cell wet at all times; (b) an electroactive soluble material (not shown) for obtaining required ionic conductivity; and, (c) a water-soluble polymer (not shown) for obtaining a required viscosity for adhering the first and second layers to the third layer.

Several preferred embodiments of the disclosed electrochemical cell include (i) engaging the electrolyte layer in a porous substance, such as, but not limited to, a filter paper, a plastic membrane, a cellulose membrane and a cloth; (ii) having the first layer of insoluble positive pole include manganese-dioxide powder and the second layer of insoluble negative pole include zinc powder; (iii) having the first layer of insoluble negative pole and/or the second layer of insoluble positive pole further include carbon powder; (iv) selecting the electroactive soluble from zinc-chloride, zinc-bromide, zinc-fluoride and potassium-hydroxide; (v) having the first layer of insoluble negative pole include silver-oxide powder and the second layer of insoluble positive pole include zinc powder and the electroactive soluble material is potassium-hydroxide; (vi) having the first layer of insoluble negative pole include cadmium powder and the second layer of insoluble positive pole include nickel-oxide powder and selecting the electroactive soluble material to be potassium-hydroxide; (vii) having the first layer of insoluble negative pole include iron powder and the second layer of insoluble positive pole include nickel-oxide powder and selecting the electroactive soluble material to be potassium-hydroxide; (viii) having the first layer of insoluble negative pole and the second layer, of insoluble positive pole include lead-oxide powder, then cell is charged by voltage applied to the poles and the electroactive soluble material is selected in this case to be sulfuric-acid; (ix) the deliquescent material and the electroactive soluble material can, be the same material such as zinc-chloride, zinc-bromide, zinc-fluoride and potassium-hydroxide; (x) the deliquescent material is selected from the group consisting of calcium-bromide, potassium-biphosphate and potassium-acetate; (xi) the water-soluble polymer can be polyvinyl alcohol, polyacrylamide, polyacrylic acid, polyvinylpyrolidone, polyethylenoxide, agar, agarose, starch, hydroxycthylcellulose and combinations and copolymers thereof; (xii) the water-soluble polymer and the deliquescent material can be the same material such as dextrane, dextranesulfate and combinations and copolymer thereof. Optionally, electroactive insoluble material includes one of or a mixture of silver, silver/silver chloride, graphite, manganese dioxide, platinum, carbon, graphite, zinc, nickel, gold and copper. Preferably, electrochemical cell 50 includes poles of carbon and zinc film. An electrochemical cell may preferably incorporate any one or more of the embodiments described above. Preferred configurations for electrochemical cells according to the present invention involve those combinations, which are devoid of poisonous compounds.

In some embodiments, the power source may be applied using a suitable printing technique.

FIG. 5 shows a schematic view of one embodiment of a powered treatment device of the present invention including a conductive interface media 100. Device 100 includes a non-power source device electrode 110 in electrical connection 125 with a power source 120 disposed on a substrate base layer 140 and a conductive interface media, such as, but not limited to a conductive gel 130 disposed in a suitable way on active device electrode 150. In the embodiment depicted in FIG. 5, power source electrode 150 is the active power source device electrode. In the example shown in FIG. 5, conductive interface media 130 is disposed on the power source electrode 150. In some embodiments conductive interface media 130 may be disposed on both the active power source device electrode 150 and the non-power source device electrode 110. In one embodiment, wherein power source includes a power source substrate base layer 132 as described in FIG. 3, conductive interface media 120 may be disposed on substrate base layer 132. Optionally, conductive interface media 130 can be disposed directly on the active electrode 150. In one non-limiting embodiment the conductive interface media 130 can be applied to the body region, which is to be treated. Optionally, device 100 can include a holding component 170. The holding component 170 is configured to facilitate accommodating the conductive interface media 130 and/or an active formulation.

In order to avoid repetition, device 100 components, which are similar to device 10 components and which were previously described for FIGS. 1a-1c will not be described again.

Conductive interface media 130 may optionally be any suitable conductive or semi-conductive composition/fluid, such as an aqueous gel, hydrogel or a conductive adhesive. Conductive composition/fluid 130 will generally be “pharmaceutically acceptable” or “physiologically acceptable” formulations for cosmetic or therapeutic use. In some embodiments, the conductive interface media is electrically conductive and adhesive hydrogel, suitable for use as a skin contact adhesive and, particularly, suitable for use as an electrical interface for electrodes of medical devices.

Optionally, the hydrogel or other conductive substance can be anhydrous or in a dehydrated state. In such an embodiment, water can be added prior to use.

In some embodiments conductive interface media 130 can include at least one additional formulation, which can optionally include active ingredients, such as drugs, ions, salts, additives, inks for tattoos or other materials known in the art of cosmetics and pharmaceutics.

The device of the present invention may be used to deliver almost any active substance/drug into/onto/through a body area, such as skin, hair, tooth, mucous membrane, nail and combination thereof. The term ‘active substance’ as used herein includes, but is not limited to any ‘active formulation’, ‘active composition’, ‘active agent’, pharmaceutical, drug, cosmeceutical, cosmetic substance, decorative substance, such as tattoo ink, moisture, water, therapeutic substance, natural and synthetic, which has an effect on any condition, such as, but not limited to a physical, physiological, biochemical, biological, chemical condition or a combination thereof. The term includes a therapeutic effect, cosmetic effect, an inhibitory effect, stimulatory effect, physical effect, biological effect, physiological effect, preventative effect, placebo effect or combination thereof. This includes therapeutic substances in all of the major therapeutic areas including, but not limited to, antiinfectives such as antibiotics and antiviral agents, analgesics including fentanyl, sufentanil, buprenorphine and analgesic combinations, anesthetics, anorexics, antiarthritics, antiasthmatic agents such as terbutaline, anticonvulsants, antidepressants, antidiabetic agents, antidiarrheals, antihistamines, antiinflammatory agents, antimigraine preparations, antimotion sickness preparations such as scopolamine and ondansetron, antinauseants, antineoplastics, antiparkinsonism drugs, cardiostimulants such as dobutamine, antipruritics, antipsychotics, antipyretics, antispasmodics; including gastrointestinal and urinary, anticholinergics, sympathornimetics, xanthine derivatives, cardiovascular preparations including calcium channel blockers such as nifedipine, beta-blockers, beta-agonists such as salbutamol and ritodrine, antiarrythmics, antihypertensives such as atenolol, ACE inhibitors, diuretics, vasodilators, including general, coronary, peripheral and cerebral, central nervous system stimulants, cough and cold preparations, decongestants, diagnostics, hormones such as parathyroid hormone, growth hormone and insulin, hypnotics, immunosuppressives, muscle relaxants, parasympatholytics, parasympathomimetics, anti-oxidants; nicotine, prostaglandins, psychostimulants, sedatives and tranquilizers, herbal preparations and homeopathic remedies.

The treatment device of the present invention is useful for the delivery of cosmetic and cosmeceutical substances, into the skin. Such substances, include, for example, skin acting anti-oxidants, such as caretenoids, ascorbic acid (vitamin C) and vitamin E, as well as other vitamin preparations and other anti-oxidants; anti wrinkling agents such as retinoids, including retinol (vitamin A alcohol), peeling agents, such as alpha-hydroxic acids, beta-hydroxy acid, better known as salicylic acid, combination-hydroxy acids and poly-hydroxy acids, and hydrolyzed and soluble collagen and others; moisturizers such as hyaluronic acid and others; anticellulite agents and acetyl-hexapeptide-3 (Argireline), pentatpeptide-3, palmitoyl-tetrapeptide-3, GHK, Myoxinol LS, N6 and Boswellic acids, caffeine and skin whitening agents such as arbutin.

It is understood that the invention may be used for delivery of a wide range of dosages of the above listed and other substances over a desired duration of time.

Active substances for the treatment of skin disorders of dermatological nature may be selected from the group comprising antibiotic, antibacterial, antifungal, antiviral, anesthetic, analgesic, antiallergic, corticosteroid, retinoid, anti-histamine, sulfur, immunosuppressant and antiproliferative medications, and mixtures thereof at any proportion. The concentration of the active substances may be adopted to exert a therapeutic effect on a disease when applied to an afflicted area.

Examples of skin disorders and other conditions of cosmetic nature are set forth in the following list: aging skin, dry skin, sun damaged skin, wrinkles, age spots, various hyperpigmented spots, melasma, puffy eyes, acne, redness of the skin, telangiectasia, cellulite, and obesity

Examples of skin disorders of dermatological nature, as well as active substances which may be used to treat them, are set forth in Table 1.

TABLE 1
A non-exhaustive listing of dermatological disorders, suitable
for usage of the iontophoretic system of the present invention
and exemplary drugs for such disorders.

| | Exemplary Active |
|---|---|
| Dermatological Disorder | Substance |
| Dermatitis | Steroidal and non-steroidal |
| Contact Dermatitis | anti-inflammatory agents |
| Atopic Dermatitis | |
| Seborrheic Dermatitis | |
| Nummular Dermatitis | |
| Chronic Dermatitis Of The Hands And Feet | |
| Generalized Exfoliative Dermatitis | |
| Stasis Dermatitis | |
| Bacterial Infections Of The Skin | Antibiotic and anti- |
| Cellulitis | inflammatory agents |
| Acute Lymphangitis | |
| Lymphadenitis | |
| Erysipelas | |
| Cutaneous Abscesses | |
| Necrotizing Subcutaneous Infections | |
| Staphylococcal Scalded Skin Syndrome | |
| Folliculitis | |
| Furuncles | |
| Hidradenitis Suppurativa | |
| Carbuncles | |
| Paronychial Infections | |
| Erythrasma | |
| Fungal Skin Infection | Antifungal agents |
| Infections caused by dermatophytes--fungi | |
| that invade only dead tissues of the | |
| skin or its appendages (stratum | |
| corneum, nails, hair) | |
| onychomycosis | |
| Infections of skin (usually of moist, | |
| occluded, intertriginous areas), skin | |
| appendages, or mucous membranes caused | |
| by yeasts of the genus Candida. | |
| Viral Skin Infection | Antiviral agents |
| Warts | |
| Herpes | |
| Disorders of the Hair Follicles And | Keratolytic agents |
| Sebaceous Glands | antibiotics |
| Acne | |
| Rosacea | Anti-inflammatory agents |
| Perioral Dermatitis | Sulfur |
| Hypertrichosis | |
| Alopecia | |
| Pseudofolliculitis Barbae | |
| Keratinous Cyst | |
| Scaling Papular Diseases | Steroidal and non-steroidal |
| Psoriasis | anti-inflammatory agents |
| Pityriasis Rosea | Anti-proliferative agents |
| Lichen Planus | |
| Pityriasis Rubra Pilaris | |
| Pigmentation Disorders | Melanin synthesis |
| Hypopigmentation | inhibitors and enhancers |
| Hyperpigmentation | |
| Scars | Retinoids (e.g., retinoic |
| | acid) |
| | Alpha and beta hydroxy |
| | acids |
| Warts | Keratolytic agents |
| Benign Tumors | Keratolytic agents |
| Moles | Antibiotics |
| Dysplastic Nevi | Anti-inflammatory agents |
| Skin Tags | |
| Lipomas | |
| Angiomas | |
| Pyogenic Granuloma | |
| Seborrheic Keratoses | |
| Dermatofibroma | |
| Keratoacanthoma | |
| Keloid | |
| Malignant Tumors | Various anticancer agents |
| Actinic keratosis (pre-cancer condition) | Photodynamic therapy |
| Basal Cell Carcinoma | agents and precursors (e.g., |
| Squamous Cell Carcinoma | porphirins and ALA) |
| Malignant Melanoma | Nonsteroidal anti- |
| Paget's Disease Of The Nipples | inflammatory drugs |
| Kaposi's Sarcoma | (NSAID) |

In some embodiments, conductive interface media 130 readily facilitates providing a conductive interfacing layer between the body/skin and active electrode and can be configured as a conductive adhesive facilitating attachment of the device to the skin.

Optionally, holding component 170 can include a retainer/substrate made of a porous non-conductive material, such as, but not limited to a sponge, pad, paper, non-woven polypropylene etc, that serves to retain the conductive interface media therein. One non-limiting example of a holding component 170 is a hydrogel, which can accommodate an active formulation. In an embodiment wherein holding component is a pad, pad can be made from any suitable material, such as from non-woven material, such as but not limited to a mixture of polyester and viscose.

In one embodiment, holding component 170 may only include a conductive formulation and an active substance can be applied as a separate layer optionally on the holding component or directly on the body area region to be treated.

Optionally, a conductive adhesive can be disposed on the counter electrode to facilitate adhering to the body area region to be treated. Conductive adhesive can be a hydrogel.

Optionally, a releasable liner (not shown in FIG. 5) can be disposed on holding component 170. Releasable liner can be removed before use. Optionally, device 100 can include a loading hole or other means for loading conductive interface media (not shown in figure), which can be used to load the conductive fluid 130 and/or active formulation before use of the device 100.

The device of the present invention can be a fully integrated device or can be part of a kit.

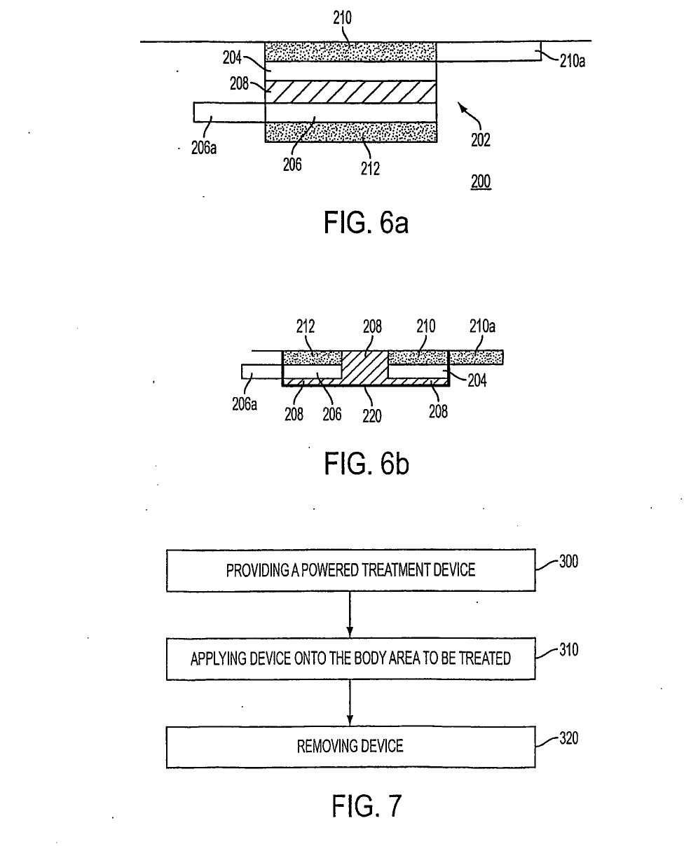

FIG. 6a shows an alternative embodiment of the treatment device of the present invention wherein two components of the power source are configured as power source treatment device electrodes. As can be seen from FIG. 6a, treatment device 200 comprises a power source 202. In some embodiments, treatment device can comprise a plurality of power sources 202. The embodiment shown in FIG. 6a is of a cofacial stacked arrangement of components, however this is for illustration purposes only and the present invention also includes an embodiment with a coplanar power source component arrangement as shown in FIG. 6b. Power source 202 comprises a negative pole 204, a positive pole 206 and an electrolyte 208 in contact with both the negative pole 204 and positive pole 206. In some embodiments, power source may include at least one current collector. FIG. 6a shows a negative current collector 210 on which is disposed the negative pole 204 and a positive current collector 212 on which is disposed the positive pole 206. Power source components which can be configured as power source treatment device electrodes include positive current collector, tabs/terminals, positive pole, negative current collector, negative pole and a conductive substrate base layer. In one embodiment the power source components which may be configured as power source treatment device electrodes may be made from biocompatible and non-toxic materials. In the embodiment shown in FIG. 6a, negative current collector 210 includes an extended region 210a. Extended negative current collector region 210a may be configured as a power source treatment device electrode. In the embodiment shown in FIG. 6a, positive pole 206 includes an extended region 206a. Extended positive pole region 206a may be configured as a second power source treatment device electrode. In an alternative embodiment which is not shown in FIG. 6a and FIG. 6b, negative current collector 210 and positive current collector 212 or extensions thereof may be configured as the two power source treatment device electrodes. The materials of the power source components are the same as described for FIGS. 1-5.

In an alternative embodiment, the power source components configured as treatment device electrodes may not include extended regions, but may include another configuration which allows dual function of the power source components. Examples include use of a power source base layer substrate, which has conductive means in the region of the dual functioning power source component. Non-limiting examples of suitable conductive means are described herein above for the embodiments wherein only one component is configured as a treatment device electrode.

Each of the power source components configured as treatment device electrodes can be configured as active/main electrodes, or counter electrodes or one active/main electrode and one counter electrode.

In one embodiment device 200 can further include a conductive interface media/composition for providing a conductive interface with a body region, such as a conductive hydrogel. The conductive composition can be an integral part of the device disposed on at least one of the device electrodes or can be part of a kit and applied directly onto the body area or attached to the device 200 before use. The device 200 can further include at least one active compound as described hereinabove.

In an embodiment as described in FIGS. 6a and 6b a power source serves dual functions as a power source and also as the electrodes of a powered treatment device, eliminating the need for any other components and facilitating optimal connection of the treatment device electrodes to the power source. Such a device obviates the need for production and assembly of separate patch electrodes, electrode connection means, battery cover and a patch frame, facilitating a cheaper production method.

FIG. 7 shows a flowchart of a method of treatment with a device of the present invention according to one embodiment, wherein one power source component is configured as a device electrode. The flowchart applies to a method of use of a fully integrated device including conductive interface media, for promoting delivery of an active substance. A device according to the present invention is provided 300 and may be applied to body region to be treated 310. In an embodiment, wherein the device includes a protective liner, protective liner may be removed from the device.

The subject may contact a body area to be treated with the electrically powered device. In such a way the non-power source device electrode and power source device electrode will be coupled to a body area. In some embodiments, electrically powered device is a thin and flexible device, which conforms to the contours of the body and which includes attachment means, for ready attachment to the body area to be treated.

In some embodiments, the contact of the device with the body area facilitates current flow and promotes delivery of active agent and body area treatment. Body area region can optionally be treated by electrical stimulation and by active agent.

Combination treatments of active substance delivery, electrical stimulation and moisturizing effect, can be possible in an embodiment wherein the anode is active, with an active drug disposed in a holding means thereon and the cathode is a counter electrode with hydrogel disposed thereon.

The device may be removed from the body area at the end of treatment time 320. Time of treatment can vary. The device is in some embodiments removed from contact with the body area after a time period, which can optionally be predetermined or is determined according to the desired dosage, the time it takes for the electrode to be depleted, or until sufficient effect or no more improvement can be seen or when a timer stops the device.

In some embodiments a pretreatment can be applied prior to use of the device. Non-limiting examples of pretreatments include applying a cleanser, applying a moisturizing composition, applying a formulation comprising a pharmaceutically active ingredient, applying a formulation comprising a cosmetically active ingredient, an anaestheic pretreatment, peeling, UV treatment, heat treatment, cold treatment, acupuncture treatment or a combination thereof.

In some embodiments a post treatment can be applied to the body area after application of the device. Non-limiting examples of post treatments include applying an occlusion formulation, applying a cleanser, applying a moisturizing composition, applying a formulation comprising a pharmaceutically active ingredient, applying a formulation comprising a cosmetically active ingredient, an anaestheic pretreatment, peeling, UV treatment, heat treatment, cold treatment, acupuncture treatment or a combination thereof.

The treatment can optionally be a one-time treatment or can be repeated in suitable time intervals any suitable number of times. Use of the present invention can facilitate temporary alleviation and elimination of the above conditions. Duration of effect can be affected by time and frequency of application, dose of active agent, type and amount of current used and severity of condition. In one embodiment, the device is a dermal patch configured for home use. In other embodiments, the device can be applied in a supervised environment.

Treatment according to the present inventions may be beneficial in all body areas. Being thin, flexible and versatile in shape and form, the devices of the present invention can be designed to fit any area of the body and to have any desirable size, according to the area having the disorder.

The treatment device of the present invention can be made by any suitable method. In an embodiment wherein the power source is a thin and flexible and printable power source, the device can be made by a printing method. The power source, which includes a power source component configured as a treatment device electrode, may be applied to a treatment device base layer substrate. In one embodiment, the power source may be made directly on at least one treatment device base layer substrate. A non-power source electrode may be applied onto at least one treatment device base layer substrate and electrically coupled to the power source. Applying may be done using a printing technique. Electrical connection can be by any suitable connection means including printed connection means.

In an embodiment, wherein a plurality of power source components are configured as both device electrodes, the step of providing a device substrate layer may not be needed and the step of connection of electrodes to the power source may not be needed. In such an embodiment, production of the device may comprise only the step of making the dual function power source.

The present invention can facilitate a miniature powered treatment device and is particularly useful for treatment of small regions, such as for example in the treatment of nail fungal infection.

One skilled in the art can appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. A treatment device comprising at least one power source for supplying electrical energy to the device, wherein at least one component of the power source is also configured as at least one power source treatment device electrode to facilitate electrical contact with a body region,
wherein the at least one power source further comprises a power source electrolyte, and
wherein the at least one component of the power source is selected from the group consisting of at least one negative pole electrode, at least one positive pole electrode, at least one current collector, an extension of a component of the power source and a combination thereof.

2. (canceled)

3. The treatment device of claim 1, wherein the at least one component of the power source comprises two power source treatment device electrodes.

4. The treatment device of claim 1, further comprising at least one non-power source device electrode of opposite polarity from the power source treatment device electrode, wherein the at least one non-power source device electrode is electrically coupled to the at least one power source to facilitate electrical contact with a body area.

5. (canceled)

6. The treatment device of claim 1, wherein the at least one power source further comprises at least one base layer substrate, at least one current collector, at least one positive pole, and at least one negative pole, wherein the at least one positive pole and at least one negative pole are disposed in spaced relation to each other in a cofacial configuration or a coplanar configuration.

7. (canceled)

8. (canceled)

9. The treatment device of claim 4, wherein the at least one component is a dual functioning power source component and the device further comprises the at least one base layer substrate comprising conductive means in the region of the dual functioning power source component that allows dual function of the dual functioning power source component, the conductive means being selected from the group consisting of a conductive substrate material, a non-conductive material comprising at least one conductive via and a combination thereof.

10-17. (canceled)

18. The treatment device of claim 1, wherein the at least one power source is a zinc/Manganese dioxide battery.

19. (canceled)

20. The treatment device of claim 1, wherein the at least one component is made from a biocompatible material.

21. The treatment device of claim 1 further comprising at least one active substance, wherein the device is configured to promote delivery of the at least one active substance into/onto the body region to be treated.

22. The treatment device of claim 1, for treatment and prevention of at least one of the group consisting of acne, skin aging, wrinkles, wounds, viral infection, bacterial infection, fungal infection, cellulite, onychomycosis, hair growth disorders, skin and teeth whitening, cleansing pores, reducing the size and/or appearance of pores, skin peeling, removing blackheads, sebum regulation acne treatment, sebum regulation, rosacea, age spots, dermatitis, skin and nail viral, fungal and bacterial infections, onychomycosis, disorders of the hair follicles and sebaceous glands, scaling disease, dark rings under the eyes, scars, wounds, pigmentation disorders, sun damaged skin, fine facial lines, laugh lines, dry skin, puffy eyes, lifting skin, folliculitis, dermatitis, psoriasis, warts, benign tumors, malignant tumors, pain management, bone healing, facilitating muscle contraction, promoting metabolic processes, increasing blood flow, treating hyperhidrosis, body decoration, vaginal candidiasis and vaginosis, genital herpes and a combination thereof.

23. The treatment device of claim 4, wherein the at least one non-power source device electrode is integrally formed with the power source.

24. (canceled)

25. The treatment device of claim 23, wherein the at least one non-power source device electrode is printed onto the power source.

26. (canceled)

27. A method of production of a treatment device of claim 1 comprising:
providing at least one substrate base layer;
applying the at least one power source onto the at least one substrate base layer.

28. The method of claim 27 further comprising applying a non-power source electrode onto the at least one base layer substrate and coupling the non-power source electrode to the at least one power source.

29. (canceled)

30. The method of claim 27, wherein applying the power source further comprises applying an extension of at least one of at least one current collector, at least one positive pole and at least one negative pole.

31. The method of claim 27, wherein applying is done using a printing technique.

32. (canceled)

33. The treatment device of claim 1, wherein the at least one component of the power source comprises at least one of a current collector or an extension of a component of the power source.

34-36. (canceled)
